(19) United States
(12) Plant Patent Application Publication
(10) Pub. No.: US 2015/0195971 P1
(43) Pub. Date: Jul. 9, 2015

(54) Pear tree named 'PremP003'

(50) ;

(71) Applicant: Allan G. White, Havelock North (NZ)

(72) Inventor: Allan G. White, Havelock North (NZ)

(21) Appl. No.: 14/544,403

(22) Filed: Dec. 31, 2014

Related U.S. Application Data
(60) Provisional application No. 61/924,150, filed on Jan. 6, 2014.

(51) Int. Cl. A01H 5/00

(52) U.S. Cl. PLT/176

(57) ABSTRACT

A new and distinct pear tree named ‘PremP003’ is disclosed. The new pear tree is notable for the size and quality of its fruit.

### LATIN NAME OF THE GENUS AND SPECIES OF THE PLANT CLAIMED

Pyrus bretschneideri×Pyrus communis

### VARIETY DENOMINATION

‘PREMP003’

### BRIEF DESCRIPTION OF THE PHOTOGRAPHS

FIG. 1 shows the fruit of a ‘PremP003’ pear tree;

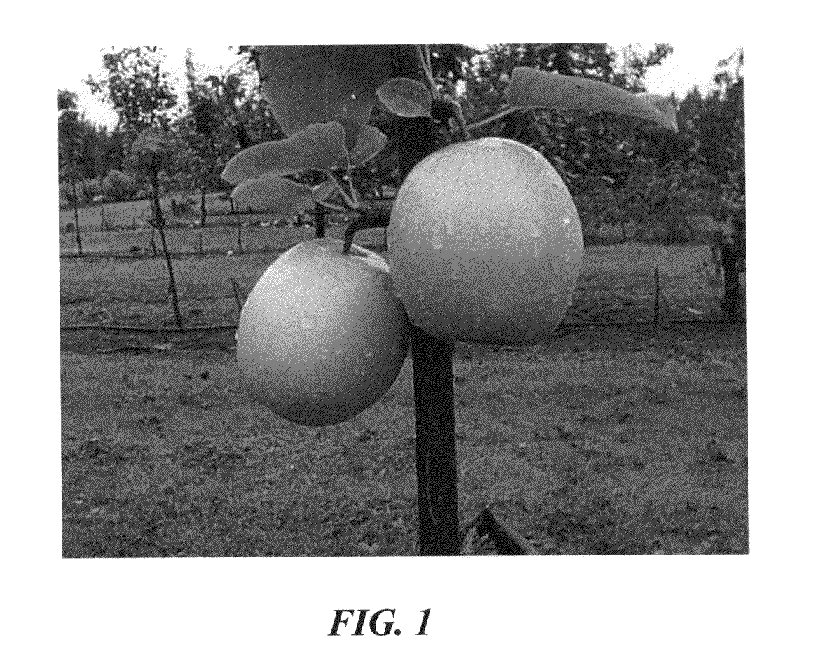

FIG. 2 shows a ‘PremP003’ pear tree;

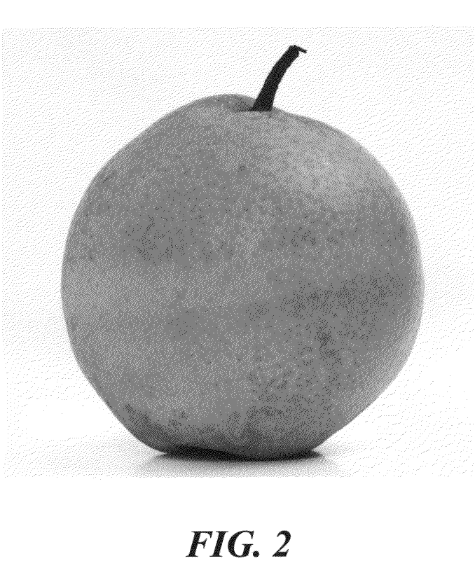

### BRIEF DESCRIPTION OF THE VARIETY

The new variety of pear tree ‘PremP003’ was created in the course of a planned breeding program carried out at Motueka, New Zealand. ‘PremP003’ originated as a result of a controlled cross of ‘Max Red Bartlett’ (seed parent, U.S. Plant Pat. No. 741) and ‘Snowflake’ (pollen parent, not patented) in 1989. ‘PremP003’ was selected in 1996 as a single plant from among a population of seedlings derived from the parents, and was selected for its fruit size and quality of fruit. Asexual propagation by budding was first carried out in Havelock North, New Zealand. The variety has since been observed over a number of asexually propagated generations, and has been found to remain true to type.

### DETAILED BOTANICAL DESCRIPTION OF THE VARIETY

The following detailed botanical description is based on observations made during the 2013 growing season at Parker, Wash. on 5 year old trees. All colors are described according to The Royal Horticultural Society Colour Chart as published in 2001. It should be understood that the characteristics described will vary somewhat depending upon cultural practices and climatic conditions, and can vary with location and season. Quantified measurements are expressed as an average of measurements taken from a number of individual plants of the new variety. The measurements of any individual plant, or any group of plants, of the new variety may vary from the stated average.
Tree: Vigor — Medium; Habit — Upright; Size — Height 9 feet, spread 1.5 feet cm; Trunk — Diameter at 5 cm above graft union 7 cm; Bark texture — smooth.
Branch: Size — Diameter 7 cm at 74 cm above soil; Crotch angle relative to trunk — 80°.
Dormant one-year-old shoot: Size — diameter 3 cm, length 13 cm; Color — 152B; Internode length — 5-6 cm; Lenticels — quantity 8 per square inch, color N155C, diameter 1 cm.
Flowers: Bud — Quantity per spur 8-9; Size — diameter 1 cm, length 1.5 cm, color — 65B, pink on edges; Petal — Quantity per flower 5; Touching, diameter 1.5 cm, length 2 cm, margin smooth; Sepals — Quantity per flower 5, diameter 0.2 cm, length 0.5 cm; Pedicel — Diameter 0.1 cm, length 2.5 cm, color 145A; Anthers — Quantity per flower 10, length 0.1 cm, pollen color 4C; Stigma — Size 0.1 cm, color 200D; Style — Length 0.4 cm, color 4C; Ovary — Size 0.3 cm, color 151B; Bloom period — First bloom April 15, full bloom April 20 in Parker, Wash.
Leaf: Attitude in relation to shoot — upward; Blade margin — serulate; Size — length 8 cm, width 5 cm; Color — upper surface 144A; Color of lower surface — 146D; Petiole length — 5 cm, diameter — 0.1 cm; Color of petiole — 176C.
Fruit: Size — diameter 8.9 cm, length 10 cm; Ribbing absent; Size of eye 3 cm; Depth of eye basin 1 cm; Width of eye basin 1.5 cm; Stalk — length 5.5 cm, width at limb attachment 0.5 cm, color N167B; Depth of stalk cavity 0.3 cm; Width of stalk cavity 0.5 cm; Size of lenticels 0.1 cm; Color of lenticels 200D; Ground color of skin 153D; Quantity per cluster 3 — 4; Flesh is crisp and juicy; Flesh color 155B; Quantity of seeds 6; Width of seed 0.4 cm; Length of seed 1 cm; Seed color Black 200A.
Use: Fresh Market.

1. We claim a new and distinct pear tree substantially as shown and described herein.
